(19) United States
(12) Patent Application Publication
(10) Pub. No.: US 2014/0296354 A1
(43) Pub. Date: Oct. 2, 2014

(54) SYNTHESIS OF PEPTOID-BASED SMALL MOLECULAR GELATORS FROM MULTIPLE COMPONENT REACTIONS

(71) Applicants: Guijun WANG, Norfolk, VA (US); Hari P. R. MANGUNURU, Norfolk, VA (US)

(72) Inventors: Guijun WANG, Norfolk, VA (US); Hari P. R. MANGUNURU, Norfolk, VA (US)

(73) Assignee: OLD DOMINION UNIVERSITY RESEARCH FOUNDATION, Norfolk, VA (US)

(21) Appl. No.: 14/242,170

(22) Filed: Apr. 1, 2014

Related U.S. Application Data
(60) Provisional application No. 61/807,035, filed on Apr. 1, 2013.

(51) Int. Cl. C07C 233/78; A61K 47/18; C07C 233/38; C07C 231/14; C07B 57/00; C07C 233/36; C07H 19/01; B01J 31/02

(52) U.S. Cl. CPC C07C 233/78; C07H 19/01; A61K 47/18; B01J 31/0247; C07C 231/14; C07B 57/00; C07C 233/36; C07C 233/38; B01J 31/0271; C07C 2101/16
USPC 514/777; 564/155; 564/158; 560/39; 560/41; 536/17.2; 514/788; 514/785; 502/167; 564/124; 530/344; 530/427; 536/23.1; 536/123.1; 516/102

(57) ABSTRACT

Described herein are the one-pot synthesis and characterization of a library of low molecular weight peptoid compounds that are able to form gels at room temperature. The compounds are synthesized from biologically-based starting materials, are biocompatible, and are resistant to degradation by proteases and peptidases. The compounds and gels synthesized therefrom can be used in such applications as tissue engineering, drug delivery, separation of biomolecules, and stimulus-responsive advanced materials

### CROSS REFERENCE TO RELATED APPLICATIONS

This application claims priority upon U.S. provisional application Ser. No. 61/807,035, filed Apr. 1, 2013. This application is hereby incorporated by reference in its entirety for all of its teachings.

### ACKNOWLEDGMENTS

The research leading to this invention was funded in part by the National Science Foundation, Grant No. CHE 1153129. The U.S. Government has certain rights in this invention.

### BACKGROUND

Gels formed via the self-assembly of molecules in solvents are becoming increasingly important in fields such as enzyme immobilization, organocatalysis, tissue engineering, and drug delivery. However, current gels used in each of these types of application suffer from limitations and/or drawbacks. New gel materials are thus needed. Low molecular weight gelators (LMWGs) are small molecules that can self-assemble and form 3-dimensional supramolecular structures that allow the trapping or immobilization of solvents. Because of the collective weak noncovalent interactions, the resulting gels formed by LMWGs are reversible and are often referred to as supramolecular gels or physical gels. Depending on the solvents used to make the gels, they can be defined as organogels for organic solvents and hydrogels for water.

LMWGs have gained considerable amount of interest due in part to their connection with supramolecular chemistry and potential applications as advanced soft materials in biomedical and materials research.¹⁻⁵ Supramolecular gels are reversible and many functional groups can be incorporated into the gelators to afford new materials with desired properties.⁶⁻⁷ For instance, organogelators have been explored as optical electronic devices and found applications in semiconductors and photovoltaic cells.^{2,8-9} They have also been explored as sequester agents for oil and chemical spill cleanup.¹⁰⁻¹² Moreover, supramolecular hydrogels have been explored more and more for biochemical and biomedical applications, as matrices for cell growth, enzyme immmolization, drug delivery, and tissue engineering.¹¹⁻¹⁵

The structures of organogelators and hydrogelators encompass a broad range of functional groups. Carbohydrates, especially monosaccharides, contain chiral centers that can be functionalized specifically to produce advanced self-assembling materials.¹⁶ When used as templates for gelators, they are also more likely to produce biocompatible gels and possess potential biomedical applications.¹⁷⁻²³ For example, the sugar based hydrogelator 1 was used to prepare semi-wet peptide/protein arrays that are compatible with enzyme activity assays and the screening of enzyme inhibitors.^{5,24} Compound 2 is a glucosamine derivative that can gel water and the resulting hydrogels have exhibited wound healing properties.²⁵

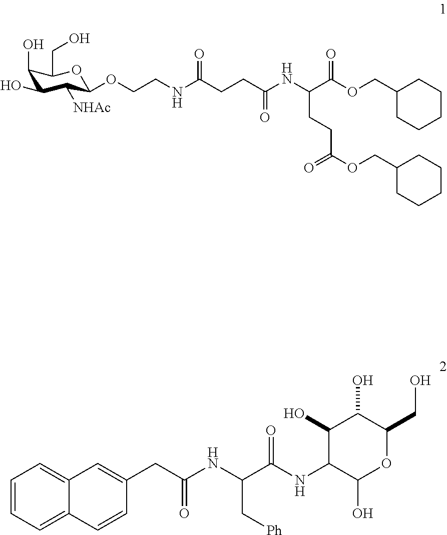

Molecular gels can be useful in which the traditional polymer hydrogels are used, with the advantage of controlling the molecular structures and incorporate functional groups into the molecules. The driving force for polymer gels are covalent forces and typically the gels are more stable, however they lack structural flexibility.

For example, enzyme immobilization is an important process in industry; reaction products can be conveniently removed from immobilized enzymes and the enzymes can then be re-used. However, current techniques for enzyme immobilization generally involve entrapment of the enzyme molecules in beads and/or adsorption of the enzymes onto inert materials. Both entrapment and adsorption can result in blockage or partial blockage of enzyme active sites, resulting in lower efficiency. New methods for enzyme immobilization, wherein active sites are fully accessible to substrates, would reduce costs and increase output of enzyme reaction products.

Alternatively, organocatalysis has become increasingly popular. It is an environmentally-friendly catalysis technique that does not require heavy metals and which can even be used for asymmetric (chiral) catalysis. However, the types of reactions that can be catalyzed by organocatalysts are limited. The ability to catalyze new and/or different reactions with organocatalysts would be of great value to the field of green chemistry.

Tissue engineering holds promise in the medical field for uses ranging from healing injuries to growing replacement organs in a laboratory setting. Gels and gelators hold promise in tissue engineering applications such as delivery of nutrients to, as scaffolds for the support of, and protection of nascent and/or healing tissues.

In drug delivery, gels are already used for the controlled release of topical medications. However, many current gelating materials and compounds are known skin irritants. Further, other drawbacks such as breakdown of the gel structure at physiological conditions and/or poor penetration of the protective layer of the skin are also associated with drug delivery gels.

One approach to the problem of making gelating compounds for medical and other applications has been to use peptide-based hydrogelators. While peptide-based compounds are indeed biocompatible, they can be degraded by proteolytic enzymes and rapidly cleared from the body. Non-biocompatible gelators, on the other hand, may be toxic and/or difficult to excrete from the body. Thus, a need exists for biocompatible and environmentally-friendly gelators for use in industry and medicine.

### SUMMARY

Described herein are the one-pot synthesis and characterization of a library of low molecular weight peptoid compounds that are able to form gels at room temperature. The compounds are synthesized from biologically-based starting materials, are biocompatible, and are resistant to degradation by proteases and peptidases. The compounds and gels synthesized therefrom can be used in such applications as tissue engineering, drug delivery, separation of biomolecules, and stimulus-responsive advanced materials.

The advantages of the invention will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows the structure of exemplary starting materials used to produce compounds having formula I.

FIG. 2 shows exemplary compounds having formula I.

FIG. 3 shows optical micrographs of gels formed by compounds 18 (a), 26 (b), and 21 (c-f). Solvent for 1(a-d) is DMSO:H₂O (1:2); solvent for 1(e-f) is EtOH:H₂O (1:2).

FIG. 4 shows the structures of starting materials used to prepare the compounds described herein.

FIG. 5 shows the structures of the compounds produced by the starting materials in FIG. 4 by the methods described herein.

FIG. 6 shows the removal of protective groups to prepare the compounds described herein.

### DETAILED DESCRIPTION

Before the present compounds, compositions, and/or methods are disclosed and described, it is to be understood that the aspects described below are not limited to specific compounds, synthetic methods, or uses, as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an,” and “the” include plural referents unless the context clearly dictates otherwise. Thus, for example, reference to “a solvent” includes mixtures of two or more such solvents, and the like.

“Optional” or “optionally” means that the subsequently-described event or circumstance can or cannot occur, and that the description includes instances where the event or circumstance occurs and instances where it does not.

As used herein, the term “about” is used to provide flexibility to a numerical range endpoint by providing that a given value may be “a little above” or “a little below” the endpoint without affecting the desired result.

“Self-assembly” as used herein refers to a process wherein molecules in solution spontaneously adopt a specific arrangement without the need of additional components or reagents. Depending upon the selection of starting materials, self-assembly can occur at room temperature or at elevated temperatures.

“Noncovalent forces” or “noncovalent interactions” describe types of intermolecular associations wherein covalent bonds are not broken or formed. Examples of noncovalent interactions include hydrogen bonding, hydrophobic interactions, π-π stacking, van der Waals interactions, cation-π interactions, anion-π interactions; C—H-π interactions, electrostatic interactions, and the like. Noncovalent interactions can typically be disrupted by altering solution conditions such as, for example, pH, salt concentration, solute concentration, temperature, solvent, and combinations thereof.

As used herein, a “gel” is a dilute cross-linked system that does not flow in the steady state. A gel typically behaves like solid due to the cross-linked network in the liquid matrix, although gels contain a large proportion of liquid by weight. In one aspect, gel structure may be due to physical (i.e., noncovalent) interactions such as intermolecular forces. In some aspects, gels are from about 0.1 to about 20% (w/v) gelator in liquid, from about 0.1 to about 10% (w/v) gelator in liquid, or from about 0.1 to about 5% (w/v) gelator in liquid. “Gelation” as used herein is the process or mechanism of forming a gel.

A “hydrogel” is a specific type of gel wherein composed of water and the gelator. Hydrogels are usually highly absorbent and may be composed of natural or synthetic polymers as well as small molecular gelators. Hydrogels are also quite flexible and may be used in such applications as, for example, tissue engineering, drug delivery, disposable diapers, contact lenses, glue, and other applications.

The “minimum gelation concentration” (MGC) is the smallest concentration at which a compound will self-assemble into a noncovalent gel structure. An MGC can be defined at any temperature and in any solvent conditions under which a compound will gel.

“Low molecular weight gelators” (LMWG) are small molecules that can self-assemble in solvents or solvent systems to form fibrous supramolecular architectures that immobilize the solvents (i.e., form gels). In one aspect, the LMWG has a molecular weight less than 3,000 Da.

As used herein, “peptidomimetic” compounds are short chains that have been designed to mimic peptides. Peptidomimetic systems include peptoids, β-peptides, and modified peptides. Peptidomimetic compounds exhibit many similar properties as peptides (e.g. secondary and tertiary structure formations, noncovalent interactions, self-assembly, and the like) but may be more resistant to degradation by peptidases and proteases due to their non-natural backbone structures.

“Peptoids” are peptidomimetic molecules that have peptide-like backbones but that have side chains attached to the backbone nitrogens instead of, or in addition to, side chains attached to the backbone α-carbons.

“Proteases” are enzymes that catabolize proteins via hydrolysis of peptide bonds in the protein backbones. Many types of proteases are known, including serine proteases, threonine proteases, cysteine proteases, aspartate proteases, glutamic proteases, and metalloproteases.

“Biocompatible” compounds, compositions, and materials do not cause adverse reactions in the body when said compounds, compositions, and materials are placed into contact with cells and/or tissues.

In the “Ugi reaction,” a ketone or an aldehyde reacts with a primary amine to form an imine or Schiff base, followed by the addition of a carboxylic acid and an isocyanide to produce a bis-amide. The exact mechanism of the Ugi reaction has not been elucidated. In one aspect, the Ugi reaction can be performed as a one-pot synthesis.

In a “multiple component reaction,” three or more chemical compounds react to form a single product. Multiple component reactions can be performed in one pot and proceed in a sequential manner, resulting in predictable products. The Ugi reaction is an example of a multiple component reaction.

“Admixing” or “admixture” refers to the combination of two or more components together so that there is no chemical reaction or physical interaction. The terms “admixing” and “admixture” also include the chemical reaction or physical interaction between or among any of the components described herein upon mixing to produce the composition. The components can be admixed alone, in water, in an organic solvent, or in a combination of solvents.

The term “aryl” or “aromatic” refers to a carbon-based group that features a delocalized conjugated π electron system with 4n+2π electrons and a coplanar ring structure, wherein n is 0 or a non-negative integer. Aromatic groups include benzene, naphthalene, and the like. “Aryl” or “aromatic” also refers to “heteroaryl” groups, which are defined as aromatic groups having at least one heteroatom incorporated within the ring structure. Examples of heteroatoms include, but are not limited to, nitrogen, oxygen, sulfur, and phosphorus. The aryl group can be substituted or unsubstituted. If substituted, substituents can include, for example, alkyl, alkynyl, alkenyl, aryl, halide, nitro, amino, ester, ketone, aldehyde, hydroxy, carboxylic acid, or alkoxy substituents or combinations thereof.

“Aliphatic” as used herein includes alkane and cycloalkane radicals, as well as alkene and alkyne radicals. The term “alkyl” refers to, unless stated otherwise, straight or branched hydrocarbon radicals, such as methyl, ethyl, n-propyl, isopropyl, n-butyl, isobutyl, t-butyl, pentyl, sec-pentyl, hexyl, heptyl, octyl, decyl, tetradecyl, hexadecyl, eicosyl, tetracosyl, and the like. Examples of longer chain alkyl groups include, but are not limited to, an oleate group or a palmitate group. A “lower alkyl” group is an alkyl group containing from 1 to 6 carbon atoms.

Alkyl groups can either be unsubstituted or can be substituted with one or more substituents, e.g. halogen, alkoxy, aryl, arylalkyl, aralkoxy, and the like. Alkyl groups include, for example, 1 to 25 carbon atoms, 1 to 8 carbon atoms, or 1 to 4 carbon atoms. An “alkyl” group as used herein can also include a cycloalkyl group, which is a non-aromatic, carbon-based ring composed of at least three carbon atoms. Examples of cycloalkyl groups include, but are not limited to, cyclopropyl, cyclobutyl, cyclopentyl, cyclohexyl, and the like. “Heterocycloalkyl groups” in which at least one of the carbon atoms of the ring is substituted with a heteroatom such as, but not limited to, nitrogen, oxygen, sulfur, or phosphorus, are also contemplated.

Alkene or alkenyl radicals are unsaturated compounds containing at least one carbon-carbon double bond and may be linear or cyclic. Alkenyl groups include, for example, ethenyl, propenyl, butadienyl, butenyl, cyclohexenyl, vinyl, allyl, and the like, and can be substituted or unsubstituted as described above. Alkyne or alkynyl radicals contain at least one triple bond between two carbon atoms and include groups such as, for example, ethynyl, propynyl, butynyl, and the like.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also to include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

Disclosed are materials and components that can be used for, can be used in conjunction with, can be used in preparation for, or are products of the disclosed compositions and methods. These and other materials are disclosed herein, and it is understood that when combinations, subsets, interactions, groups, etc., of these materials are disclosed, that while specific reference of each various individual and collective combination and permutation of these compounds may not be explicitly disclosed, each is specifically contemplated and described herein. For example, if an aldehyde is disclosed and a number of different amines are discussed, each and every combination and permutation of aldehyde and amine that is possible is specifically contemplated unless specifically indicated to the contrary. For example, if a class of molecules A, B, and C are disclosed, as well as a class of molecules D, E, and F, and an example of a combination A+D is disclosed, then even if each is not individually recited, each is individually and collectively contemplated. Thus, in this example, each of the combinations A+E, A+F, B+D, B+E, B+F, C+D, C+E, and C+F, are specifically contemplated and should be considered disclosed from disclosure of A, B, and C; D, E, and F; and the example combination of A+D. Likewise, any subset or combination of these is also specifically contemplated and disclosed. Thus, for example, the sub-group of A+E, B+F, and C+E is specifically contemplated and should be considered disclosed from disclosure of A, B, and C; D, E, and F; and the example combination of A+D. This concept applies to all aspects of this disclosure including, but not limited to, steps in methods of making and using the disclosed compositions. Thus, if a variety of additional steps that can be performed with any specific embodiment or combination of embodiments of the disclosed methods, and that each such combination is specifically contemplated and should be considered disclosed.

References in the specification and concluding claims to parts by weight of a particular element or component denote the weight relationship between the element or component and any other elements or components in the compound or composition for which a part by weight is expressed. Thus, in a compound containing 2 parts by weight of component X and 5 parts by weight of component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

Variables such as R¹, R², R³, R⁴, R⁵, and so forth, are used throughout the application and are the same variables as previously defined unless stated to the contrary.

### Peptoid-Based Small Molecular Gelators and Preparation Thereof

In one aspect, a method is provided wherein an Ugi reaction is performed wherein an amine, a carbonyl compound (i.e. a ketone or aldehyde), a carboxylic acid, and an isocyanide are admixed to generate a compound of formula (I):

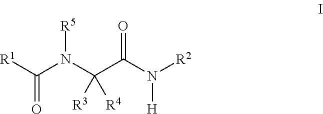

In one aspect, the method involves reacting the following components:

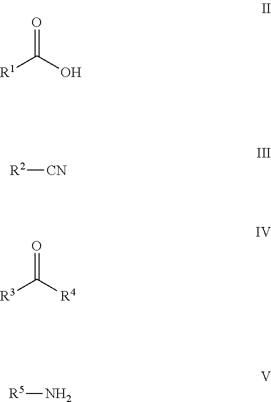

In another aspect, the method involves admixing components II-V in the following order:
(a) admixing compound V with compound IV to produce a first admixture;
(b) admixing compound II with the first admixture to produce a second admixture; and
(c) admixing compound III with the second admixture to produce the compound having the formula I.

Each variable in I-V above as well as procedures for making the compounds of formula I are discussed in detail below.

In certain aspects, R¹ is hydrogen; a branched or straight chain C₁ to C₂₅ alkyl, alkenyl, or alkynyl group; a cycloalkyl or heterocycloalkyl group; a substituted or unsubstituted aryl group; or a heteroaryl group; R² is a cycloalkyl or heterocycloalkyl group; an alkyl ester of a carboxylic acid; a carboxylic acid; a substituted or unsubstituted aryl group; R³ and R⁴ is, independently, hydrogen; a branched or straight chain C₁ to C₂₅ alkyl, alkenyl, or alkynyl group, which may be substituted or unsubstituted; a cycloalkyl or heterocycloalkyl group; a substituted or unsubstituted aryl group; or a heteroaryl group; and R⁵ is a branched or straight chain C₁ to C₂₅ alkyl, alkenyl, or alkynyl group, which may be substituted or unsubstituted; an amino sugar or a protected amino sugar; a cycloalkyl or heterocycloalkyl group; a substituted or unsubstituted aryl group; or a heteroaryl group.

In a further aspect, R¹ is an alkyl group, an aryl group, an alkynyl group, or a substituted aryl group. In a further aspect, R¹ is a methyl group, a butynyl group, a naphthyl group (with or without a methylene group at any position of the naphthyl group), an unsubstituted or substituted phenyl group (e.g., bromo, nitro), or a benzyl group and the benzyl group optionally is a substituent, and the substituent is a bromo group or a nitro group.

In one aspect, R1 is derived from carboxylic acid derivative a glucosamine or a protected glucosamine. For example, the carboxylic moiety can be covalently bonded to the amino group of glucosamine via a tether. The tether can be an alkyl group, a cycloalkyl or heterocycloalkyl group; a substituted or unsubstituted aryl group; or a heteroaryl group. In one aspect, a diacid or anhydride can be reacted with glucosamine to link the carboxylic moiety to glucosamine. In one aspect, R¹ has the formula

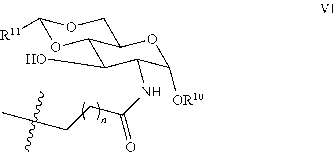

wherein R¹° and R¹¹ are an alkyl or aryl group as defined herein, and n is from 1 to 5. In this aspect, the R¹ group is protected. The protective groups can be removed using techniques known in the art to produce a group having the formula below.

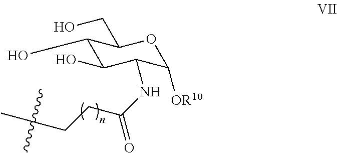

In certain aspects, R² is a cycloalkyl group or an alkyl ester of a carboxylic acid. In a further aspect, R² is a cyclohexyl group or ethyl aceto (formula VIII below).

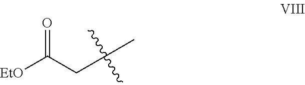 [SMILES: *CC(=O)OCC]

In another aspect, R² is

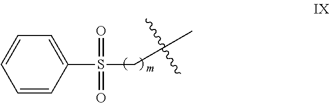

wherein m is from 1 to 5, and the phenyl group can be unsubstituted or substituted with one or more groups (e.g., alkyl).

In some aspects, R³ and R⁴ is hydrogen. In another aspect, R³ is hydrogen and R⁴ is a substituted or unsubstituted aryl group (e.g., phenyl group substituted with one or more alkoxy groups).

In certain aspects, R⁵ is an aryl group, a substituted aryl group, an alkyl group, an amino sugar, or a protected amino sugar. In a further aspect, R⁵ is a glucosamine, a protected glucosamine, an α-methylbenzyl group, or a substituted or unsubstituted benzyl group. In one aspect, the benzyl group is substituted with an alkoxy group such as, for example, a methoxy group.

In one aspect, equimolar amounts of each reactant (i.e. amine V, carbonyl IV, carboxylic acid II, and isocyanide III) are used as starting materials. Each component used to prepare compounds having formula I is discussed in detail below.

“Amines” as used herein are organic compounds containing at least one amino group. In one aspect, the amines are primary amines and include one carbon-nitrogen single bond and two nitrogen-hydrogen single bonds Amines include amino acids such as, for example, glycine, alanine, serine, threonine, cysteine, valine, leucine, isoleucine, methionine, phenylalanine, tyrosine, tryptophan, aspartic acid, glutamic acid, asparagine, glutamine, histidine, lysine, arginine, ornithine, lanthionine, 2-aminoisobutyric acid, dehydroalanine, γ-aminobutyric acid, citrulline, β-alanine, and the like. Other primary amines, including but not limited to diamines, are also contemplated including ammonia, allylamine, 1,3-cyclohexanebis(methylamine), 1,3-diaminoacetone, 1,5-diamino-2-methylpentane, ethylenediamine, 1,9-diaminononane, ethylamine, ethanolamine, propylamine, butylamine, 2,2-dimethyl-1,3-propanediamine, 4,9-dioxa-1,12-dodecanediamine, 2,2′(ethylenedioxy)bis(ethylamine), methylamine, 4,7,10-trioxa-1,13-tridecanediamine, tris(2-aminoethyl)amine, xylenediamine, benzylamine, methoxybenzylamine, dimethoxybenzylamine, chlorobenzylamine, phenylethylamine, related compounds, and isomers thereof. In the case of primary amines containing an aromatic ring, heteroaromatic amines in which one or more ring atoms are hetero atoms such as, for example, sulfur, phosphorus, oxygen, or nitrogen, are also contemplated. Further, aliphatic and aromatic groups with primary amine substituents may be further substituted with, for example, halogen atoms, nitro groups, alkyl groups, and the like. Primary amines may also be formed via decarboxylation of amino acids and include compounds such as, for example, histamine and tryptamine. Amino sugars such as galactosamine, glucosamine, mannosamine, are also contemplated, as are amino sugars that have been protected and/or derivatized. In one aspect, the amine is benzylamine. In another aspect, the amine is 4-methoxybenzylamine. In a still further aspect, the amine is 1-phenylethylamine. In an alternative aspect, the amine is a protected glucosamine.

Thus, in one aspect, compounds having the formula X can be prepared from a diamine (i.e., formula V is H₂N—R⁵—NH₂), where R¹-R⁵ are defined above.

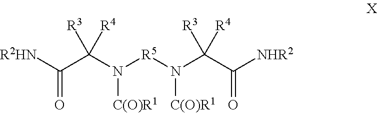

A “protected” or “derivatized” compound is a compound in which reactive groups such as, for example, hydroxyl groups, carbonyl groups, amine groups, carboxylic acid groups, and the like, have been temporarily or permanently prevented from reacting further through chemical modification. In some aspects, protection and/or derivatization are reversible and function to provide chemoselectivity in subsequent reactions. Many protecting groups are known in the art, as are techniques for their addition and removal.

“Ketones” as used herein are organic compounds containing a carbonyl group bonded to two other carbon atoms. “Ketones” as used herein also includes diketones and cyclic ketones, as well as substituted ketones. Substitutions include aliphatic and aryl groups as defined below. Examples of ketones include compounds such as, for example, acetone, ethyl methyl ketone, methyl propyl ketone, diethyl ketone, methyl butyl ketone, methyl pentyl ketone, methyl hexyl ketone, methyl heptyl ketone, methyl octyl ketone, ethyl propyl ketone, ethyl butyl ketone, ethyl pentyl ketone, ethyl hexyl ketone, ethyl heptyl ketone, acetophenone, isophorone, phorone, carvone, camphor, muscone, cyclohexanone, 2-cyclohexen-1-one, 2-cyclopenten-1-one, methyl-2-cyclohexenone, cyclopentanone, methyl-2-cyclopentenone, 2-cyclohepten-1-one, cycloheptanone, and the like. Ketones also include ketose sugars such as, for example, dihydroxyacetone, erythrulose, ribulose, xylulose, fructose, psicose, sorbose, tagatose, sedoheptulose, D-manno-octulose, D-glycero-D-galacto-nonulose.

When R³ and R⁴ in formula IV are different groups, a chiral center is produced. The two diastereoisomers can be subsequently separated using techniques known in the art to produce optically pure compounds.

“Aldehydes” as used herein are organic compounds containing one or more formyl groups. Formyl groups have the general formula R—CHO and contain carbonyl centers bonded to a) an R group (any alkyl or other carbon-containing side chain) and b) a hydrogen. “Aldehydes” as used herein also includes dialdehydes and substituted aldehydes. Substitutions include aliphatic and aryl groups as defined below. Examples of aldehydes include compounds such as, for example, formaldehyde, acetaldehyde, propionaldehyde, butyraldehyde, benzaldehyde, cinnamaldehyde, vanillin, citral, tolualdehyde, furfural, retinaldehyde, glyoxal, malondialdehyde, succindialdehyde, glutaraldehyde, and phthalaldehyde. Aldehydes also include aldose sugars in their open chain forms such as, for example, glycolaldehyde, glyceraldehyde, erythrose, threose, ribose, arabinose, xylose, lyxose, allose, altrose, glucose, mannose, gulose, idose, galactose, and talose.

An “isocyanide” is an organic compound with a functional group a) containing a triple bond between a nitrogen atom and a carbon atom and b) wherein the functional group is connected to the rest of the molecule via the nitrogen atom. Isocyanides may include, for example, butyl isocyanide, 4-methoxyphenyl isocyanide, methylbenzyl isocyanide, 1-pentyl isocyanide, 1,1,3,3-tetramethylbutyl isocyanide, benzyl isocyanide, cyclohexyl isocyanide, isopropyl isocyanide, tert-butyl isocyanide, 2,6-dimethylphenyl isocyanide, 2-pentyl isocyanide, 1-adamantyl isocyanide, p-toluenesulfonylmethyl isocyanide, 2-morpholinoethyl isocyanide, phenyl isocyanide, 2-naphthyl isocyanide, 1H-benzotriazol-1-ylmethyl isocyanide, tert-butyl 2-isocyanopropionate, di-tert-butyl 2-isocyanosuccinate, tert-butyl 2-isocyano-3-methylbutyrate, tert-butyl 2-isocyano-3-phenylpropionate, ethyl isocyanoacetate, diethyl isocyanomethylphosphonate, or mixtures thereof.

In another aspect, the isocyanide can include two or more cyano groups. Thus, in one aspect, compounds having the formula XI can be prepared from a diisocyanate (i.e., formula III is NC—R²—CN), where R¹-R⁵ are defined above.

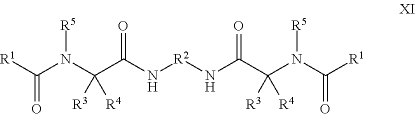

“Carboxylic acids” as used herein are organic acids characterized by the presence of at least one carboxyl group. Carboxylic acids can be aliphatic or aromatic and may contain substituted or unsubstituted carbons in addition to the carboxyl group. Substituents can include halogens, alkyl groups, alkenyl groups, alkynyl groups, nitro groups, and combinations thereof. In some aspects, the carboxylic acids are dicarboxylic acids or tricarboxylic acids. Carboxylic acids include such as, for example, formic acid, acetic acid, chloroacetic acid, dichloroacetic acid, trichloroacetic acid, trifluoroacetic acid, oxalic acid, benzoic acid, propionic acid, butanoic acid, valeric acid, caproic acid, enanthic acid, caprylic acid, pelargonic acid, capric acid, lauric acid, myristic acid, palmitic acid, stearic acid, arachidic acid, malic acid, pyruvic acid, niacin, citric acid, aconitic acid, isocitric acid, ketoglutaric acid, succinic acid, fumaric acid, oxaloacetic acid, salicylic acid, bromobenzoic acid, nitrobenzoic acid, acrylic acid, pentynoic acid, hexynoic acid, butynoic acid, propynoic acid, and the like, and mixtures thereof. Carboxylic acids also include amino acids as described above, as well as proline and sarcosine, which do not contain primary amines.

In one aspect, diacids can be used. Thus, compounds having the formula XII can be prepared from a diacid (i.e., formula II is HOOC—R¹—COOH), where R¹-R⁵ are defined above.

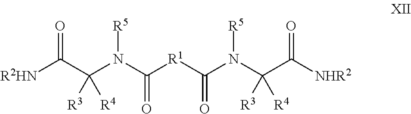

For example, the diacid can be

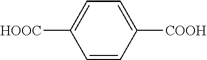 [SMILES: O=C(O)c1ccc(C(=O)O)cc1]

where R¹ in formula XII is a phenyl group.

In one aspect, the amine V and the carbonyl reactant IV are mixed in the presence of a solvent such as, for example, methanol, and stirred. Following this initial reaction, the carboxylic acid II and the isocyanide III are added and the reaction is allowed to continue. In this aspect, the reaction is monitored by a technique such as thin layer chromatography until the spots on the TLC plate corresponding to the starting materials have disappeared. The TLC plate can be visualized by any method known in the art such as, for example, staining, charring, exposure to iodine vapor, UV shadowing, or a combination thereof. Exemplary procedures for producing the compounds of formula I are provided in the Examples.

In a further aspect, the compound of formula I produced by the above reaction can be diluted, washed, and/or extracted with various solvents such as, for example, dichloromethane or water to remove side products. The solvent layer containing the product may be further dried by any technique known in the art such as, for example, with activated molecular sieves, anhydrous sodium sulfate, and the like. The dried solution containing the compound of formula (I) may be further dried or concentrated, for example, under reduced pressure as in a rotary evaporator or similar device. The concentrated crude product may be further purified by a technique such as column chromatography, flash chromatography, preparatory TLC, recrystallization, or any other technique known in the art. In some aspects, the purified product is characterized by techniques such as ¹H NMR, ¹³C NMR, mass spectrometry, melting point determination, and the like. Exemplary procedures for isolating and characterizing the compounds of formula I are provided in the Examples.

In one aspect, the compound of formula (I) is N-benzyl-N-(2-(cyclohexylamino)-2-oxoethyl)pent-4-ynamide; N-benzyl-4-bromo-N-(2-(cyciohexylainino)-2-oxoethyl)benzamide; N-benzyl-N-(2-(cyclohexylamino)-2-oxoethyl)-4-nitrobenzamide; N-(4-methoxybenzyl)-4-bromo-N-(2-(cyclohexylamino)-2-oxoethyl)benzamide; ethyl 2-(2-(N-(4-methoxybenzyl)-4-bromobenzamido)acetamido)acetate; 4-Bromo-N-(2-(cyclohexylamino)-2-oxoethyl)-N-(8-hydroxy-6-methoxy-2-phenyl-hexahydropyrano[3,2-d][1,3]dioxin-7-yl)benzamide, or ethyl-2-(2-(4-bromo-N-(8-hydroxy-6-methoxy-2-phenyl-hexahydropyrano[3,2-d][1,3]dioxin-7-yl)benzamido)acetamido)acetate.

### Preparation of Gels and Applications Thereof

The compounds having the formula I can be used to form gels having numerous applications. In one aspect, the compounds having the formula I is added to one or more solvents in order to produce a gel. “Solvent” as used herein refers to any substance—usually a liquid—that dissolves a solute (i.e., a chemically different solid, liquid, or gas) to result in a solution. In some aspects, solvents can also refer to the liquid components of gels as defined above. A solvent as used herein can be water or an organic solvent. Solvents can be nonpolar such as, for example, hydrocarbons like pentane, cyclopentane, hexane, cyclohexane, benzene, toluene, xylene, 1,4-dioxane, chloroform, or diethyl ether. In other aspects, the solvents can alternatively be polar, aprotic solvents such as, for example, dichloromethane, tetrahydrofuran, ethyl acetate, acetone, dimethylformamide, acetonitrile, pyridine, carbon disulfide, benzonitrile, or dimethyl sulfoxide. Alternatively, the solvent can be polar protic solvents such as alcohols and carboxylic acids including, but not limited to, formic acid, n-butanol, isopropanol, n-propanol, ethanol, methanol, acetic acid, ethylene glycol, propylene glycol, glycerin, or water. Mixtures of solvents can also be used herein. The term “solvent” can also refer to a mixture of two or more solvent compounds in any ratio. For example, the solvent can be a mixture of water with a hydrocarbon (e.g., hexane) or an alcohol (e.g., ethanol). In a still further aspect, the solvent is a 2:1, 1:1, or 1:2 ratio of ethanol to water. In another aspect, the solvent is a 2:1, 1:1, or 1:2 ratio of DMSO to water. When two or more solvents are used, the ratio can vary depending upon the nature of the gelator as well as the solvents.

In one aspect, the gels described herein can be formed by incrementally adding a compound of formula I to a solvent at room temperature. In other aspects, the solvent is heated and the compound of formula I is added to the heated solvent, then allowed to cool to room temperature, wherein a gel is formed. In some aspects, the admixture of solvent and compound of formula I is stirred prior to cooling to ensure thorough mixing. The amount of compound of formula I needed to make the gel can vary depending upon the solvent selected and the nature of the compound. The morphology of the gel can vary. In certain aspects, the gel is composed of a uniform, fibrous network. In one aspect, the fibers of the network have an average diameter of 0.1 to 1 μm and average length of 10 to 1,000 μm. Methods for determining and characterizing the morphology of the gels described herein are provided in the Examples.

The gels produced from the compounds having formula I have numerous applications. For example, the gels can be used to immobilize a number of different materials including bioactive agents (e.g., enzymes, pharmaceutical drugs, etc.) as well as catalysts. The term “immobilized” as used herein includes entrapping or locking the material in place within the gel. In other aspects, the gels can be used in tissue engineering, where viable cells and growth factors are immobilized in the gel. Besides cells and bioactive compounds, gels can also be used to clean up oil spill or chemical spills. Gelators with chiral components can also be used to separate amino acids or peptides or proteins based on the differences of their interaction with the gelator matrices. Certain gelators can also function as electronic device and optical devices.

The component of choice can be immobilized on the gel using techniques known in the art. In one aspect, the substance to be immobilized is added to a warm mixture gelator/solvent, and the mixture is cooled down in order for the gel to form. In other aspects, the substance can be added to the solvent together with the gelator, and the mixture heated or sonicated until a homogeneous solution is obtained, then the solution is cooled down for gelation to occur. Alternatively for enzymes or those substances that are heat sensitive, the gelator can be dissolved in an organic solvent such as, for example, ethanol or DMSO to form a homogeneous solution, then the substance is added to the solution. Afterwards, water is added to the solution at room temperature to form the gel with the immobilized substance. In another aspect, the compounds described herein are useful in separating large biomolecules which are chiral.

In one aspect, described herein is a method for separating one or more chiral compounds, the method comprising contacting the one or more chiral compounds with a gel comprising a compound described herein, wherein the compound is optically active (e.g., excess of one enantiomer over the other enantiomer). For example, compounds possessing sugar moieties (e.g., glucosamine residue) can be useful in this aspect. The gel can be used in a similar fashion as that of agarose gels that are currently used to separate large molecules. Examples of large molecules that can be separate and purified include peptides, proteins, DNA, or glycans.

### EXAMPLES

The following examples are put forth so as to provide those of ordinary skill in the art with a complete disclosure and description of how the compounds, compositions, and methods described and claimed herein are made and evaluated. The examples are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. Numerous variations and combinations of reaction conditions, e.g., component concentrations, desired solvents, solvent mixtures, temperatures, pressures, and other reaction ranges and conditions that can be used to optimized the product purity and yield obtained from the described process. Only reasonable and routine experimentation will be required to optimize such processes and conditions.

### Example 1

### General Procedure

Reagents and solvents were obtained from commercial suppliers (Sigma-Aldrich, Acros Organics, Fisher, etc.) and were used directly without any purification. Unless otherwise noted, all reactions were carried out in oven-dried glassware under a nitrogen atmosphere. Combiflash chromatography was carried out using SiliCycle 230-400 mesh silica gel. Thin layer chromatography (TLC) analysis was performed using Merck Kieselgel 60 F 254 plates and visualized using UV light and phosphomolybdic acid (PMA) staining. ¹H NMR and proton-decoupled ¹³C NMR spectra (CDCl₃ with TMS internal standard) were obtained using 400 MHz Bruker spectrometers. Products were further characterized using high resolution mass spectrometry (electrospray ionization, positive ion mode).

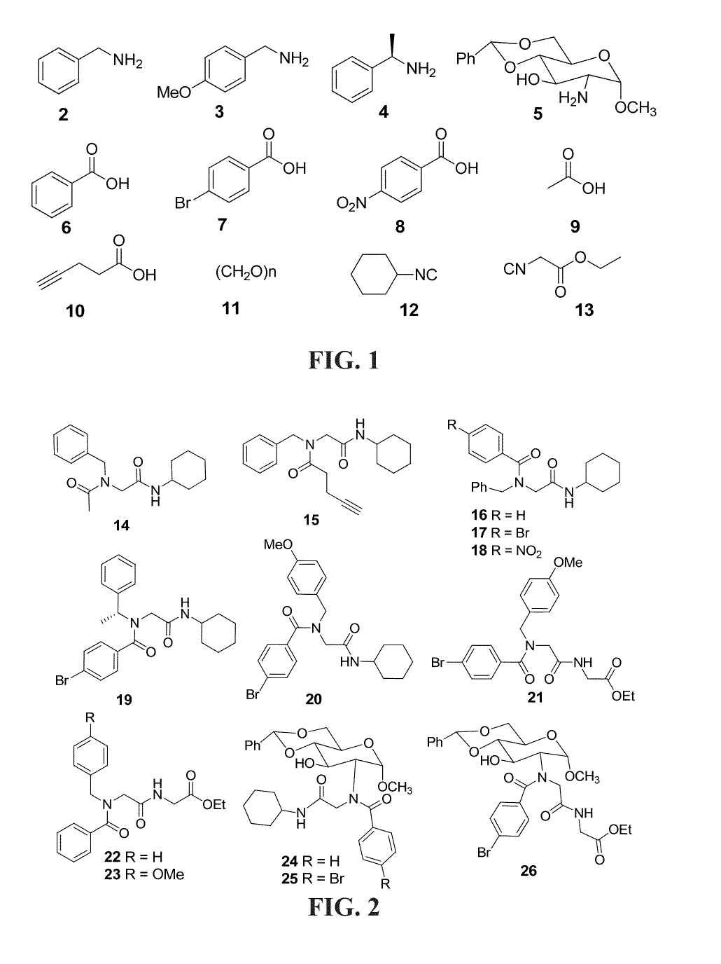

FIG. 1 shows the structure of exemplary starting materials used to produce compounds having formula I. FIG. 2 shows exemplary compounds having formula I produced herein.

Benzyl amine (50 mg, 0.46 mmol) was added to a solution of paraformaldehyde (0.013 g, 0.46 mmol) in methanol (6 mL). The solution was stirred at room temperature for 1 h. Benzoic acid (0.055 g, 0.46 mmol) was added, followed by cyclohexyl isocyanide (0.051 g, 0.46 mmol). The reaction was monitored using TLC, which indicated completion after 24 h. The reaction mixture was diluted with dichloromethane (15 mL) and washed with water (10 mL). The organic layer was then dried over anhydrous sodium sulfate and concentrated under reduced pressure. The crude product was purified by combiflash chromatography using 60% ethyl acetate in hexane. The desired product (compound 16) was obtained as a white solid (0.145 g) with 88% yield.

Other products were synthesized and purified following similar procedures.

### Example 2

### N-benzyl-N-(2-(cyclohexylamino)-2-oxoethyl)acetamide (Compound 14)

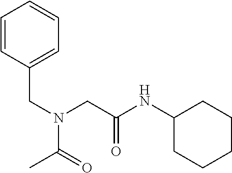 [SMILES: CC(=O)N(CC(=O)NC1CCCCC1)Cc1ccccc1]

Compound 14 was synthesized following the method of Example 1. The pure compound was obtained as a white solid with 0.112 g (83%), m.p. 122.0-124.0° C. ¹H NMR (400 MHz, CDCl₃): (mixture of rotamers) δ 0.82 (m, 2H), 1.07 (m, 4H), 1.26 (m, 4H), 1.59 (m, 7H), 1.75 (m, 3H), 2.05 (s, 1H), 2.13 (s, 3H), 3.62 (m, 1H), 3.82 (s, 1H), 3.85 (s, 2H), 4.54 (s, 1H), 4.58 (s, 2H), 5.47 (d, J=7.26 Hz, 1H), 6.15 (d, J=7.26 Hz, 1H), 7.08 (m, 2H), 7.20-7.31 (m, 6H). ¹³C (100 MHz, CDCl₃): (mixture of rotamers) δ 21.4, 21.7, 24.7, 24.8, 25.3, 25.5, 32.7, 32.8, 48.1, 48.2, 50.5, 50.7, 52.5, 53.3, 126.6, 127.9, 128.0, 128.6, 129.0, 129.1, 135.8, 137.0, 166.8, 167.9, 171.5, 171.9. HRMS (ESI+) calcd for C₁₇H₂₄N₂O₂ [M+Na]+, 311.1729. found 311.1727.

### Example 3

### N-benzyl-N-(2-(cyclohexylamino)-2-oxoethyl)pent-4-ynamide (Compound 15)

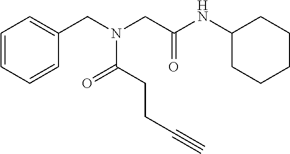 [SMILES: C#CCCC(=O)N(CC(=O)NC1CCCCC1)Cc1ccccc1]

Compound 15 was synthesized following the method of Example 1. The pure compound was obtained as a white solid with 0.109 g (71%), m.p. 138.0-140.0° C. ¹H NMR (400 MHz, CDCl₃): (mixture of rotamers) δ 0.93 (qd, J=11.8 Hz, 1H), 1.14 (m, 3H), 1.34 (m, 3H), 1.68 (m, 6H), 1.87 (m, 2H), 1.99 (m, 1H), 2.58 (m, 2H), 2.69 (m, 2H), 3.71 (m, 1H), 3.92 (s, 1H), 3.97 (s, 2H), 4.65 (s, 1H), 4.70 (s, 2H), 5.58 (d, J=8.0 Hz, 1H), 6.24 (d, J=8.0 Hz, 1H), 7.19 (d, J=7.3 Hz, 2H), 7.34 (m, 5H). ¹³C (100 MHz, CDCl₃): (mixture of rotamers) δ 14.5, 14.7, 24.7, 24.8, 25.3, 25.5, 31.9, 32.3, 32.8, 32.9, 48.1, 48.4, 50.7, 50.9, 51.5, 52.4, 69.0, 69.1, 126.5, 127.9, 128.0, 128.6, 129.0, 135.6, 136.8, 166.7, 167.7, 171.9, 172.4. HRMS (ESI+) calcd for C₂₀H₂₆N₂O₂ [M+Na]+, 349.1886. found 349.1884.

### Example 4

### N-benzyl-N-(2-(cyclohexylamino)-2-oxoethyl)benzamide (Compound 16)

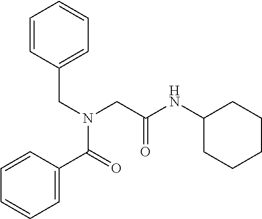 [SMILES: O=C(CN(Cc1ccccc1)C(=O)c1ccccc1)NC1CCCCC1]

Compound 16 was synthesized following the method of Example 1. The pure compound was obtained as a white solid with 0.145 g (88%), m.p. 104.0-106.0° C. ¹H NMR (400 MHz, CDCl₃): (mixture of rotamers) δ 1.10 (m, 4H), 1.28 (m, 3H), 1.60 (m, 4H), 1.78 (m, 3H), 3.66 (m, 2H), 3.96 (s, 2H), 4.57 (s, 2H), 4.72 (s, 1H), 5.59 (br, 1H), 6.29 (br, 1H), 7.11 (m, 2H), 7.22-7.41 (m, 15H). ¹³C (100 MHz, CDCl₃): (mixture of rotamers) δ 24.7, 25.5, 32.9, 48.1, 49.4, 54.0, 126.8, 127.1, 127.9, 128.6, 128.9, 130.1, 135.2, 135.9, 167.7, 172.9. HRMS (ESI+) calcd for C₂₂H₂₆N₂O₂ [M+Na]⁺, 373.1886. found 373.1882.

### Example 5

### N-benzyl-4-bromo-N-(2-(cyclohexylamino)-2-oxoethyl)benzamide (Compound 17)

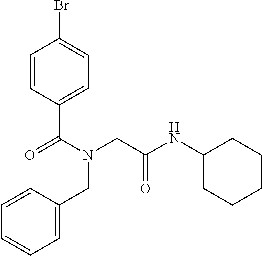 [SMILES: O=C(CN(Cc1ccccc1)C(=O)c1ccc(Br)cc1)NC1CCCCC1]

Compound 17 was synthesized following the method of Example 1. The pure compound was obtained as a white solid with 0.165 g (82%), m.p. 168.0-170.0° C. ¹H NMR (400 MHz, CDCl₃): (mixture of rotamers) δ 1.06 (m, 5H), 1.26 (m, 4H), 1.58 (m, 6H), 1.77 (m, 4H), 3.65 (m, 2H), 3.93 (s, 2H), 4.54 (s, 2H), 4.66 (s, 1H), 5.78 (br. s, 1H), 6.22 (br. s, 1H), 7.18-7.33 (m, 9H), 7.43 (d, J=7.5 Hz, 2H). ¹³C (100 MHz, CDCl₃): (mixture of rotamers) δ 24.7, 25.4, 32.9, 48.2, 48.4, 49.2, 49.7, 51.7, 54.0, 124.5, 127.0, 127.9, 128.6, 128.9, 131.8, 134.1, 135.8, 167.3, 171.8. HRMS (ESI+) calcd for C₂₂H₂₅N₂O₂Br [M+Na]+, 451.0991. found 451.0997.

### Example 6

### N-benzyl-N-(2-(cyclohexylamino)-2-oxoethyl)-4-nitrobenzamide (Compound 18)

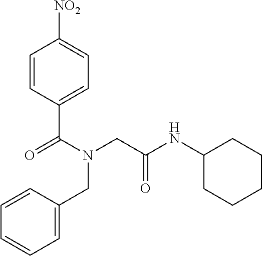 [SMILES: O=C(CN(Cc1ccccc1)C(=O)c1ccc([N+](=O)[O-])cc1)NC1CCCCC1]

Compound 18 was synthesized following the method of Example 1. The pure compound was obtained as a white solid with 0.162 g (87%), m.p. 190.0-192.0° C. ¹H NMR (400 MHz, CDCl₃): (mixture of rotamers) δ 1.10 (m, 10H), 1.67 (m, 10H), 3.59 (s, 1H), 3.68 (m, 2H), 3.98 (s, 2H), 4.52 (s, 2H), 4.73 (s, 1H), 5.22 (s, 1H), 5.89 (d, J=4.8 Hz, 1H), 7.08 (d, J=6.5 Hz, 2H), 7.28 (m, 7H), 7.58 (d, J=8.0 Hz, 2H), 7.63 (d, J=7.5 Hz, 2H), 8.17 (m, 2H). ¹³C (100 MHz, CDCl₃): (mixture of rotamers) δ 24.7, 25.3, 25.4, 25.5, 30.5, 33.0, 48.4, 48.9, 53.9, 123.9, 126.9, 127.9, 128.0, 128.1, 128.2, 128.6, 128.7, 129.0, 129.1, 135.3, 141.4, 148.6, 166.8, 170.6. HRMS (ESI+) calcd for C₂₂H₂₅N₃O₄ [M+Na]+, 418.1737. found 418.1741.

### Example 7

### (R)-4-bromo-N-(2-(cyclohexylamino)-2-oxoethyl)-N-(1-phenylethyl)benzamide (Compound 19)

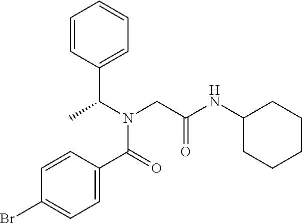 [SMILES: C[C@H](c1ccccc1)N(CC(=O)NC1CCCCC1)C(=O)c1ccc(Br)cc1]

Compound 19 was synthesized following the method of Example 1. The pure compound was obtained as a white solid with 0.131 g (71%), m.p. 140.0-142.0° C. ¹H NMR (400 MHz, CDCl₃): (mixture of rotamers) δ 1.09 (m, 3H), 1.27 (m, 3H), 1.48 (m, 2H), 1.55 (s, 2H), 1.56 (s, 3H), 1.75 (m, 2H), 3.37 (m, 2H), 3.63 (s, 3H), 4.15 (d, J=14.6 1H), 4.50 (s, 1H), 6.40 (br. s, 1H), 7.13 (br, 1H), 7.23 (m, 2H), 7.27 (m, 5H), 7.50 (m, 2H). ¹³C (100 MHz, CDCl₃): (mixture of rotamers) δ 17.9, 24.6, 25.5, 32.7, 32.8, 47.1, 48.0, 57.6, 64.2, 124.2, 126.7, 127.0, 128.0, 128.7, 128.9, 132.0, 134.7, 139.1, 168.3, 171.8. HRMS (ESI+) calcd for C₂₃H₂₇N₂O₂Br [M+Na]+, 465.1148. found 465.1156.

### Example 8

### N-(4-methoxybenzyl)-4-bromo-N-(2-(cyclohexylamino)-2-oxoethyl)benzamide (Compound 20)

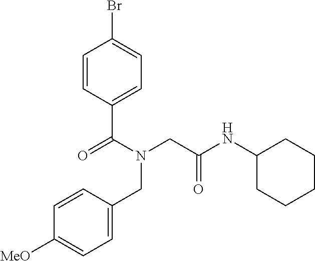 [SMILES: COc1ccc(CN(CC(=O)NC2CCCCC2)C(=O)c2ccc(Br)cc2)cc1]

Compound 20 was synthesized following the method of Example 1. The pure compound was obtained as a white solid with 0.132 g (79%), m.p. 174.0-176.0° C. ¹H NMR (400 MHz, CDCl₃): (mixture of rotamers) δ 1.09 (m, 4H), 1.29 (q, J=12.0 Hz, 3H), 1.58 (m, 5H), 1.79 (m, 3H), 3.67 (m, 2H), 3.73 (s, 3H), 3.93 (s, 2H), 4.48 (s, 2H), 4.64 (s, 1H), 6.11 (d, 1H), 6.80 (d, J=8.3 Hz, 2H), 7.00 (d, J=5.3 Hz, 2H), 7.29 (d, J=8.3 Hz, 2H), 7.46 (d, J=7.8 Hz, 2H).¹³C (100 MHz, CDCl₃): (mixture of rotamers) δ 24.71, 24.74, 25.4, 48.2, 49.2, 53.5, 55.3, 114.3, 124.5, 128.4, 128.6, 131.8, 159.4, 167.4, 171.7. HRMS (ESI+) calcd for C₂₃H₂₇N₂O₃Br [M+Na]+, 481.1097. found 481.1103.

### Example 9

### Ethyl 2-(2-(N-(4-methoxybenzyl)-4-bromobenzamido)acetamido)acetate (Compound 21)

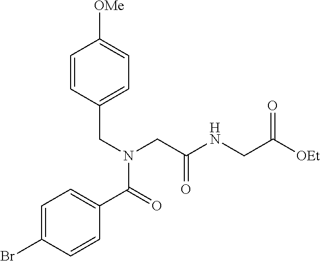 [SMILES: CCOC(=O)CNC(=O)CN(Cc1ccc(OC)cc1)C(=O)c1ccc(Br)cc1]

Compound 21 was synthesized following the method of Example 1. The pure compound was obtained as a white solid with 0.112 g (66%), m.p. 123.0-125.0° C. ¹H NMR (400 MHz, CDCl₃): (mixture of rotamers)) δ 1.23 (m, 3H), 3.82 (s, 3H), 4.02 (d, J=5.3 Hz, 2H), 4.12 (br, s, 2H), 4.24 (q, J=7.0 Hz, 2H), 4.57 (br, 2H), 6.89 (m, 2H), 7.13 (d, J=5.0 Hz, 2H), 7.43 (m, 2H), 7.56 (d, J=8.0 Hz, 2H). ¹³C (100 MHz, CDCl₃): (mixture of rotamers) δ 14.1, 41.2, 48.4, 53.4, 55.3, 61.6, 114.3, 124.5, 128.4, 128.7, 131.8, 134.0, 134.1, 158.2, 159.4, 168.6, 169.6, 171.9. HRMS (ESI+) calcd for C₂₁H₂₃BrN₂O₅[M+Na]+, 485.0682. found 485.0680.

### Example 10

### Ethyl 2-(2-(N-benzylbenzamido)acetamido)acetate (Compound 22)

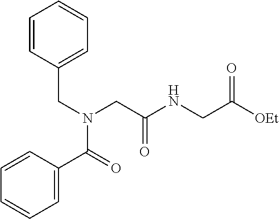 [SMILES: CCOC(=O)CNC(=O)CN(Cc1ccccc1)C(=O)c1ccccc1]

Compound 22 was synthesized following the method of Example 1. The pure compound was obtained as a light yellow oil with 0.134 g (81%). ¹H NMR (400 MHz, CDCl₃): (mixture of rotamers) δ 1.22 (m, 3H), 3.67 (s, 2H), 3.91 (d, J=3.5 Hz, 2H), 4.04 (br, s, 2H), 4.13 (q, J=7.0 Hz, 2H), 4.57 (br. s, 2H), 4.74 (s, 1H), 6.90 (s, 1H), 7.12 (s, 2H), 7.21-7.35 (m, 8H), 7.44 (m, 2H). ¹³C (100 MHz, CDCl₃): (mixture of rotamers) δ 14.2, 41.0, 41.2, 48.6, 52.3, 53.9, 61.5, 126.9, 127.1, 127.9, 128.6, 128.9, 130.2, 135.1, 135.8, 168.9, 169.6, 173.0. HRMS (ESI+) calcd for; C₂₀H₂₂N₂O₄ [M+Na]+, 377.1471. found 377.1471.

### Example 11

### Ethyl 2-(2-(N-(4-methoxybenzyl)benzamido)acetamido)acetate (Compound 23)

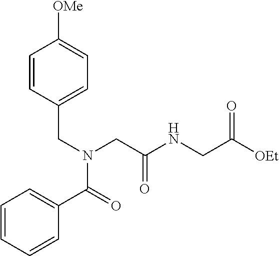 [SMILES: CCOC(=O)CNC(=O)CN(Cc1ccc(OC)cc1)C(=O)c1ccccc1]

Compound 23 was synthesized following the method of Example 1. The pure compound was obtained as a white solid with 0.094 g (67%), m.p. 94.0-96.0° C. ¹H NMR (400 MHz, CDCl₃): (mixture of rotamers) δ 1.22 (m, 3H), 3.72 (s, 3H), 3.92 (d, J=5.3 Hz, 2H), 4.04 (br, s, 2H), 4.13 (q, J=7.0 Hz, 2H), 4.57 (br, 2H), 6.79 (d, J=8.5 Hz, 2H), 6.99 (m, 1H), 7.03 (d, J=5.5 Hz, 2H), 7.34 (m, 3H), 7.44 (m, 2H). ¹³C (100 MHz, CDCl₃): (mixture of rotamers) δ 14.1, 41.2, 48.4, 53.4, 55.3, 61.5, 114.2, 126.9, 127.5, 128.3, 128.6, 130.0, 130.1, 133.1, 135.2, 159.3, 168.9, 169.6, 172.9. HRMS (ESI+) calcd for C₂₁H₂₄N₂O₅ [M+Na]+, 407.1577. found 407.1575.

### Example 12

### N-(2-(cyclohexylamino)-2-oxoethyl)-N-(8-hydroxy-6-methoxy-2-phenyl-hexahydropyrano[3,2-d][1,3]dioxin-7-yl)benzamide (Compound 24)

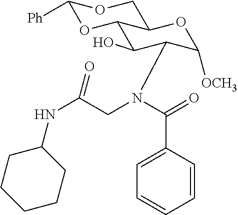 [SMILES: COC1OC2COC(c3ccccc3)OC2C(O)C1N(CC(=O)NC1CCCCC1)C(=O)c1ccccc1]

Compound 24 was synthesized following the method of Example 1. The pure compound was obtained as a white solid with 0.083 g (89%), m.p. 261.0-263.0° C. ¹H NMR (400 MHz, CDCl₃): δ 1.08 (m, 3H), 1.27 (m, 2H), 1.58 (m, 3H), 1.87 (dd, J=18.6, 14.1 Hz, 2H), 3.30 (s, 3H), 3.46 (t, J=9.2 Hz, 1H), 3.59 (m, 1H), 3.72 (m, 2H), 3.90 (dd, J=10.0, 3.5 Hz, 1H), 4.12 (m, 2H), 4.17 (d, J=4.8 Hz, 2H), 4.45 (d, J=3.5 Hz, 1H), 5.44 (s, 1H), 5.83 (d, J=8.0 Hz, 1H), 6.49 (d, J=1.5 Hz, 1H), 7.26 (dd, J=5.0, 1.8 Hz, 3H), 7.33 (d, J=8.3 Hz, 2H), 7.42 (m, 2H), 7.50 (d, J=8.3 Hz, 2H).¹³C (100 MHz, CDCl₃): δ 24.6, 25.4, 32.9, 47.6, 49.0, 55.2, 62.7, 63.3, 66.6, 68.7, 80.9, 100.8, 101.7, 124.1, 126.3, 128.2, 128.8, 129.0, 132.0, 134.0, 137.1, 169.7, 172.5. HRMS (ESI+) calcd for C₂₉H₃₆N₂O₇ [M+Na]+, 547.2414. found 547.2420.

### Example 13

### 4-Bromo-N-(2-(cyclohexylamino)-2-oxoethyl)-N-(8-hydroxy-6-methoxy-2-phenyl-hexahydropyrano[3,2-d][1,3]dioxin-7-yl)benzamide (Compound 25)

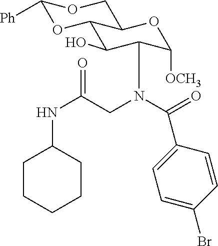 [SMILES: COC1OC2COC(c3ccccc3)OC2C(O)C1N(CC(=O)NC1CCCCC1)C(=O)c1ccc(Br)cc1]

Compound 25 was synthesized following the method of Example 1. The pure compound was obtained as a white solid with 0.098 g (91%), m.p. 291.0-293.0° C. ¹H NMR (400 MHz, CDCl₃): δ 1.1 (m, 3H), 1.26 (m, 2H), 1.53 (m, 1H), 1.62 (m, 2H), 1.87 (m, 2H), 3.31 (s, 3H), 3.48 (m, 1H), 3.62 (m, 1H), 3.73 (m, 2H), 3.84 (dd, J=9.9, 3.6 Hz, 1H), 4.15 (m, 4H), 4.44 (d, J=3.5 Hz, 1H), 5.46 (s, 1H), 5.78 (d, J=8.0 Hz, 1H), 6.49 (d, J=1.5 Hz, 1H), 7.27 (dd, J=5.0, 1.8 Hz, 3H), 7.33 (d, J=8.3, 2H), 7.42 (m, 2H), 7.50 (d, J=8.3 Hz, 2H). ¹³C (100 MHz, CDCl₃): δ 24.7, 25.4, 32.84, 32.89, 47.6, 48.9, 55.2, 62.6, 63.3, 66.7, 68.7, 80.9, 100.9, 101.7, 126.3, 127.0, 128.2, 128.7, 129.0, 129.7, 135.2, 137.1, 169.9, 173.5. HRMS (ESI+) calcd for C₂₉H₃₅N₂O₇Br [M+Na]+, 625.1519. found 625.1530.

### Example 14

### Ethyl-2-(2-(4-bromo-N-(8-hydroxy-6-methoxy-2-phenyl-hexahydropyrano[3,2-d][1,3]dioxin-7-yl)benzamido)acetamido)acetate (Compound 26)

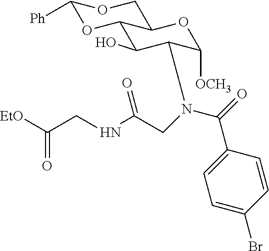 [SMILES: CCOC(=O)CNC(=O)CN(C(=O)c1ccc(Br)cc1)C1C(OC)OC2COC(c3ccccc3)OC2C1O]

Compound 26 was synthesized following the method of Example 1. The pure compound was obtained as a white solid with 0.065 g (61%), m.p. 207.0-209.0° C. ¹H NMR (400 MHz, CDCl₃): δ 1.26 (m, 3H), 3.31 (s, 3H), 3.44 (m, 1H), 3.61 (m, 1H), 3.73 (m, 1H), 3.82 (dd, J=10.0, 3.5 Hz, 1H), 3.90 (dd, J=18.3, 3.3 Hz, 1H), 4.08 (m, 2H), 4.15 (m, 4H), 4.32 (m, 2H), 4.47 (d, J=3.5 Hz, 1H), 5.44 (s, 1H), 6.05 (d, J=2.0 Hz, 1H), 6.66 (m 1H), 7.26 (dd, J=4.9, 1.9 Hz, 3H), 7.21 (d, J=8.3 Hz, 2H), 7.40 (m, 2H), 7.50 (d, J=8.3 Hz, 2H). ¹³C (100 MHz, CDCl₃): δ 14.1, 41.6, 47.0, 55.2, 61.8, 62.5, 63.3, 66.6, 68.7, 81.0, 100.7, 100.8, 124.2, 126.3, 128.2, 128.8, 129.1, 132.0, 134.0, 137.0, 169.6, 170.9, 172.6. HRMS (ESI+) calcd for C₂₇H₃₁BrN₂O₉ [M+Na]+, 629.1105. found 629.1103.

### Example 15

### Gelation Test Results

Gelation tests were performed by adding incremental amounts of compounds to a solvent or solvent system at room temperature and observing the results (i.e., gel formation, solubility of compounds, insolubility of compounds, or formation of a precipitate). The term “insoluble” means that the compound does not dissolve upon heating and sonicating, even at temperatures above the boiling points of the solvent. The term “precipation” means that the compound dissolves upon heating and sonicating, however, it crashes out of solution when the heat is removed.

Gelation test results are presented in Table 1.

TABLE 1
Gelation Test Results for Library Compounds

| Com- | | | | EtOH:H₂O | EtOH:H₂O | DMSO:H₂O | DMSO:H₂O |
|---|---|---|---|---|---|---|---|
| pound | Hexane | H₂O | EtOH | (1:1) | (1:2) | (1:1) | (1:2) |
| 14 | I^{(a)} | P | S | S | P | S | P |
| 15 | P^{(b)} | S | P | P | G 20.0 | G 20.0 | G 20.0 |
| 16 | I | I | P | P | P | P | P |
| 17 | I | P | S | G 20.0 | P | G 10.0 | G 5.0 |
| 18 | I | P | P | G 20.0 | P | G 20.0 | G 10.0 |
| 19 | S^{(c)} | P | S | P | P | G 20.0 | P |
| 20 | P | P | P | G 10.0 | P | G 5.0 | G 6.6 |
| 21 | I | S | G 20.0 ^{(d)} | G 5.0 | G 2.8 | G 2.5 | G 2.0 |
| 22 | S | S | P | P | P | S | P |
| 23 | I | S | S | P | P | P | G 20.0 |
| 24 | P | I | G 20.0 | P | G 20.0 | P | P |
| 25 | P | I | G 20.0 | G 20.0 | G20.0 | G 20.0 | P |
| 26 | I | P | I | G 20.0 | G10.0 | G 10.0 | G 6.6 |

^{(a)}I, insoluble
^{(b)}P, precipitating
^{(c)}S, soluble at ~20 mg/mL
^{(d)} G, gel at room temperature; number is minimum gelation concentration

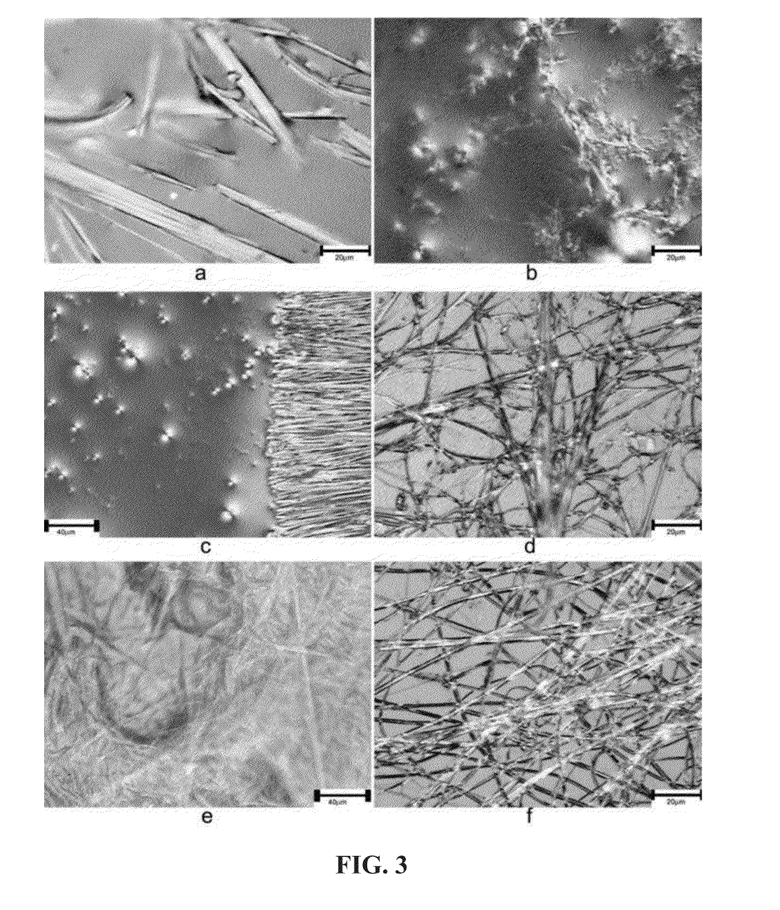

FIG. 3 shows the optical micrographs of several gels. Compound 18 formed a gel in DMSO aqueous mixture at 2 wt %, which is not a very efficient gelator, the morphology of the gel appeared as rod or sheet like feature (FIG. 3a). Compound 26 formed a gel in DMSO/H₂O (1:2) at 0.66 wt %, which is a more efficient gelator, and the gels formed thinner fibrous assembly (FIG. 3b). The compound 21 is the most efficient gelator identified in this study, interestingly the morphology of the self-assembled structures in DMSO/H₂O and EtOH/H₂O both appeared as very long and uniform fibrous assemblies. The DMSO/H₂O gel on the edge part appeared to form a cluster of aligned fibers bundled together during the drying process while in the mild of the sample showed more separated fibers (FIG. 3c, d). The EtOH/H₂O gel has shown more uniform fibrous structures with an average diameter of 0.5 μm and over 300 μm in length (FIG. 3e, f). The morphology study indicated that there is certain correlation between the gelation efficiency and the fibrous assemblies.

### Example 16

### Gel Melting Temperatures

Melting points were measured for compounds that were gelators in the DMSO:H₂O (1:2) solvent system. In general, a compound was dissolved in heated solvent in a small vial at its minimum gelation concentration and transferred into a tube (e.g., an NMR tube) while the mixture was still warm. The tube was sonicated and cooled until a stable gel formed. The tube was immersed in an oil bath and the temperature of the solid-to-liquid phase transition was monitored using a thermometer as the oil bath temperature was changed. Gel melting temperatures are presented in

TABLE 2
Table 2: Gel Melting Temperatures

| | MGC in DMSO:H₂O | T1 | T2 | T3 |
|---|---|---|---|---|
| Compound | (1:2), (mg/mL) | (° C.)^{(a)} | (° C.)^{(b)} | (° C.)^{(c)} |
| 15 | 20.0 | 95 | 100 | 110 |
| 17 | 5.0 | 78 | 80 | 90 |
| 18 | 10.0 | 91 | 95 | 110 |
| 20 | 6.6 | 91 | 110 | 120 |
| 21 | 2.0 | 70 | 75 | 81 |
| 23 | 20.0 | 50 | 52 | 58 |
| 26 | 6.6 | 110 | 118 | 128 |

^{(a)}Temperature at which gel begins to melt.
^{(b)}Temperature at which gel is approximately half melted.
^{(c)}Temperature at which gel is fully melted.

### Example 17

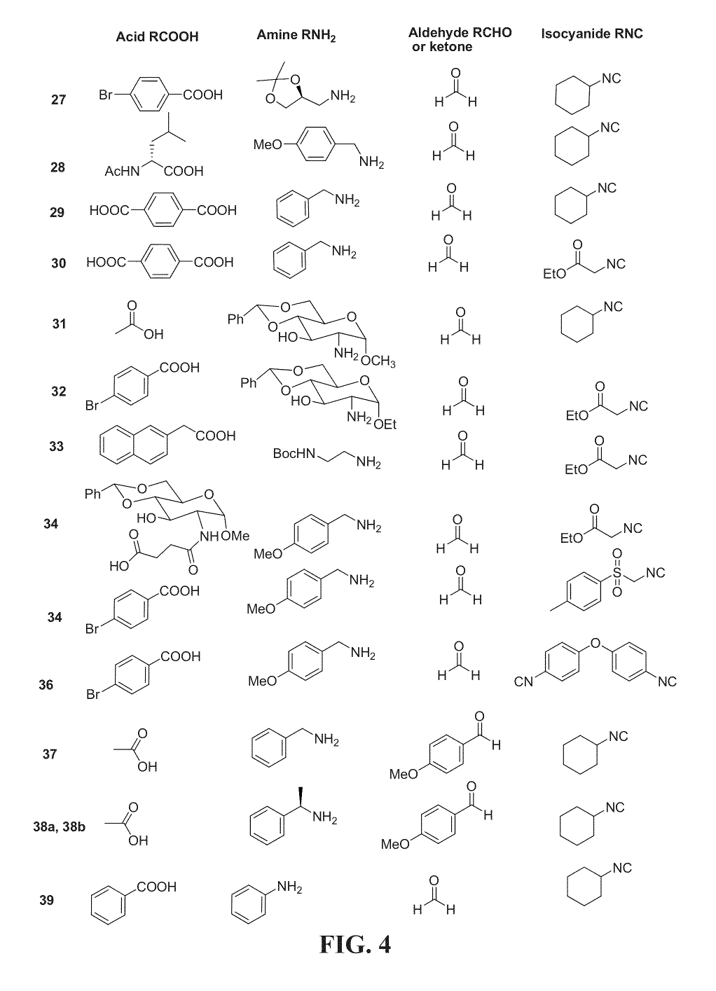

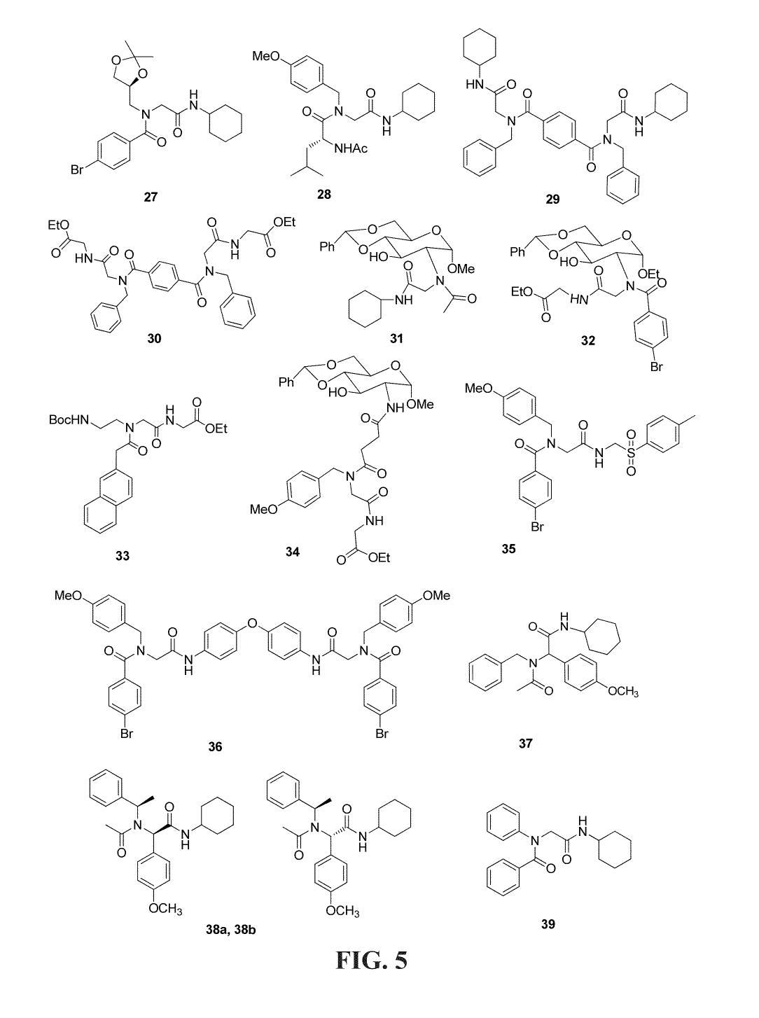

Multiple component reactions were performed by the methods described herein. Using the starting materials in FIG. 4, the reactions proceeded smoothly with isolated yields 58-90%, with the lower yields are for the diacid substrates. Structures of compounds 27-39 are provided in FIG. 5. Either a pair of enantiomers (entry 37) or diastereomers (entry 38) were obtained in about 90% total yields. The diastereomers can be separated via flash chromatography on silica gel and they are labeled as 38a and 38b.

After the compounds were synthesized, they were screened for gelation properties. As shown in Table 3, many of these peptoids can form gels in DMSO/water mixture. Compound 34 provided the best results, which was able to gelate in water as well as almost all the tested solvents. For the compounds with new chiral centers, they formed gels in aqueous environment preferentially.

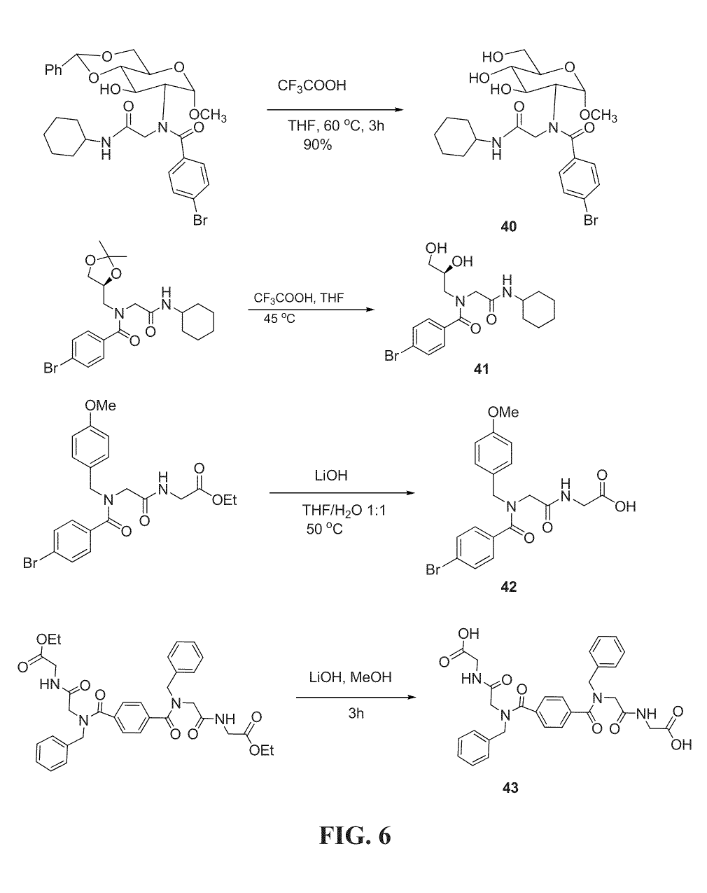

Besides the direct Ugi reactions, several products of the Ugi reaction were converted to other compounds as shown in FIG. 6. Typically a hydrophobic functional group is removed from the product in the hope to improve hydrogen bonding capacity with aqueous solutions. These compounds were also screened for their effectiveness as organo/hydrogelators. However, unfortunately these compounds did not show enhanced gelation when compared to their precursors.

TABLE 3

| Com- | | | | DMSO:H₂O | DMSO:H₂O | EtOH:H₂O |
|---|---|---|---|---|---|---|
| pound | Hexane | Water | EtOH | 1:1 | 1:2 | (1:2) |
| 27 | S | S | P | G 20.0 | G 20.0 | P |
| 28 | P | UG 20.0 | S | UG 20.0 | UG 20.0 | P |
| 29 | I | UG 20.0 | UG | P | G 20.0 | P |
| 30 | I | I | S | P | P | P |
| 31 | I | P | G 10.0 | G 10.0 | G 10.0 | S |
| 32 | I | P | S | G 20.0 | UG 20.0 | G 20.0 |
| 33 | I | P | S | P | P | P |
| 34 | I | G 10.0 | G 10.0 | G 20.0 | G 10.0 | G 20.0 |
| 35 | I | I | P | G 20.0 | G 20.0 | G 10.0 |
| 36 | I | P | S | UG 20.0 | P | P |
| 37 | I | UG17.0 | S | ND | P | G 15.0 |
| 38a | P | UG17.0 | S | ND | UG18.0 | I |
| 38b | ND | G 21.0 | ND | ND | I | P |
| 39 | P | P | S | P | P | P |
| 40 | S | I | S | G 20.0 | G 20.0 | P |
| 41 | I | S | S | S | S | S |
| 42 | I | P | S | P | P | P |
| 43 | I | P | S | UG 20.0 | UG 20.0 | ND |

S, soluble (starting test at 20 mg/mL);
I, insoluble;
P, precipitate;
G, stable gel, the numbers after G are preliminary MGCs in mg/mL;
UG, slightly unstable gels or partial gels, concentrations are 20 mg/mL unless otherwise noted;
ND, not determined.

Throughout this publication, various publications are referenced. The disclosures of these publications in their entireties are hereby incorporated by reference into this application in order to more fully describe the methods, compositions, and compounds herein.

Various modifications and variations can be made to the methods, compositions, and compounds described herein. Other aspects of the methods, compositions, and compounds will be apparent from consideration of the specification and practice of the methods, compositions, and compounds disclosed herein. It is intended that the specification and examples be considered as exemplary.

### REFERENCES

1. Yu, L.; Ding, J. Chem. Soci. Rev. 2008, 37, 1473-81.
2. Yagai, S.; Kitamura, A. Chem. Soci. Rev. 2008, 37, 1520-9.
3. Sangeetha, N. M.; Maitra, U. Chem. Soci. Rev. 2005, 34, 821-36.
4. Lyon, L. A.; Meng, Z.; Singh, N.; Sorrell, C. D.; St John, A. Chem. Soci. Rev. 2009, 38, 865-74.
5. Zhao, F.; Ma, M. L.; Xu, B. Chem. Soci. Rev. 2009, 38, 883-91.
6. Hirst, A. R.; Escuder, B.; Miravet, J. F.; Smith, D. K. Angew. Chem., Int. Ed. 2008, 47, 8002-18.
7. (a) Ajayaghosh, A.; Vijayakumar, C.; Varghese, R.; George, S. J. Angew. Chem., Int. Ed. 2006, 45, 456-60; (b) Ajayaghosh, A.; Praveen, V. K.; Vijayakumar, C.; George, S. J. Angew. Chem., Int. Ed. 2007, 46, 6260-5.
8. Ajayaghosh, A.; Praveen, V. K.; Vijayakumar, C. Chem. Soci. Rev. 2008, 37, 109-22.
9. Dastidar, P. Chem. Soci. Rev. 2008, 37, 2699-715.
10. Jadhav, S. R.; Vemula, P. K.; Kumar, R.; Raghavan, S. R.; John, G. Angew. Chem. Int. Ed. Engl. 2010, 49, 7695-8.
11. Zhao, F.; Ma, M. L.; Xu, B. Chem. Soc. Rev. 2009, 38, 883-91.
12. Zhou, S. L.; Matsumoto, S.; Tian, H. D.; Yamane, H.; Ojida, A.; Kiyonaka, S.; Hamachi, I. Chemistry 2005, 11, 1130-6.
13. Bowerman, C. J.; Nilsson, B. L. Am. Chem. Soc. 2010, 132, 9526-7.
14. Gao, Y.; Kuang, Y.; Guo, Z.-F.; Guo, Z.; Krauss, I. J.; Xu, B. J. Am. Chem. Soc. 2009, 131, 13576-7.
15. Bhuniya, S.; Seo, Y. J.; Kim, B. H. Tetrahedron Lett. 2006, 47, 7153-6.
16. Vemula, P. K.; John, G. Acc. Chem. Res. 2008.
17. (a) Vemula, P. K.; Cruikshank, G. A.; Karp, J. M.; John, G. Biomaterials 2009, 30, 383-93; (b) Wang, W.; Wang, H.; Ren, C.; Wang, J.; Tan, M.; Shen, J.; Yang, Z.; Wang, P. G.; Wang, L. Carbohydr. Res. 2011, 346, 1013-7.
18. Liao, S. W.; Yu, T.-B.; Guan, Z. J. Am. Chem. Soc. 2009, 131, 17638-46.
19. Yang, Z.; Liang, G.; Wang, L.; Xu, B. J. Am. Chem. Soc. 2006, 128, 3038-43.
20. Shome, A.; Debnath, S.; Das, P. K. Langmuir 2008, 24, 4280-8.
21. (a) Wu, D.-Q.; Wang, T.; Lu, B.; Xu, X.-D.; Cheng, S.-X.; Jiang, X.-J.; Zhang, X.-Z.; Zhuo, R.-X. Langmuir 2008, 24, 10306-12; (b) Yang, Z.; Liang, G.; Xu, B. Soft Matter 2007, 3, 515-20.
22. Saez, J. A.; Escuder, B.; Miravet, J. F. Tetrahedron 2010, 66, 2614-8.
23. Jung, J. H.; Rim, J. A.; Cho, E. J.; Lee, S. J.; Jeong, I. Y.; Kameda, N.; Masuda, M.; Shimizu, T. Tetrahedron 2007, 63, 7449-56.
24. Kiyonaka, S.; Sada, K.; Yoshimura, I.; Shinkai, S.; Kato, N.; Hamachi, I. Nat. Mater. 2004, 3, 58-64.
25. Yang, Z.; Liang, G.; Xu, B. Acct. Chem. Res. 2008, 41, 315-26.
26. Wang, G.; Cheuk, S.; Williams, K.; Sharma, V.; Dakessian, L.; Thorton, Z. Carbohydr. Res. 2006, 341, 705-16.
27. Wang, G.; Yang, H.; Cheuk, S.; Coleman, S. Beilstein J. Org. Chem. 2011, 7, 234-42.
28. Goyal, N.; Cheuk, S.; Wang, G. Tetrahedron 2010, 66, 5962-71.
29. Wang, G.; Cheuk, S.; Yang, H.; Goyal, N.; Reddy, P. V. N.; Hopkinson, B. Langmuir 2009, 25, 8696-8705.
30. Nie, X.; Wang, G. J. Org. Chem. 2006, 71, 4734-41.
31. Cheuk, S.; Stevens, E. D.; Wang, G. Carbohydr. Res. 2009, 344, 417-25.

What is claimed:

1. A compound having the formula: 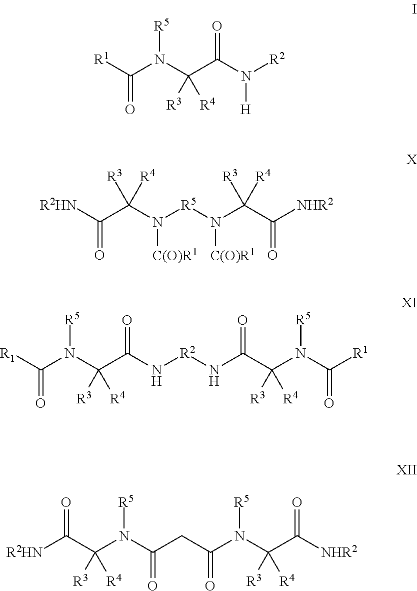
wherein
R¹ is hydrogen; a branched or straight chain C₁ to C₂₅ alkyl, alkenyl, or alkynyl group; a cycloalkyl or heterocycloalkyl group; a substituted or unsubstituted aryl group; or a heteroaryl group;
R² is a cycloalkyl or heterocycloalkyl group; an alkyl ester of a carboxylic acid; or a carboxylic acid;
R³ and R⁴ are, independently, hydrogen; a branched or straight chain C₁ to C₂₅ alkyl, alkenyl, or alkynyl group, which may be substituted or unsubstituted; a cycloalkyl or heterocycloalkyl group; a substituted or unsubstituted aryl group; or a heteroaryl group; and
R⁵ is a branched or straight chain C₁ to C₂₅ alkyl, alkenyl, or alkynyl group, which may be substituted or unsubstituted; an amino sugar or a protected amino sugar; a cycloalkyl or heterocycloalkyl group; a substituted or unsubstituted aryl group; or a heteroaryl group.

2. The compound of claim 1, wherein R¹ is an alkyl group, an aryl group, an alkynyl group, or a substituted aryl group.

4. The compound of claim 1, wherein R¹ is an unsubstituted or substituted phenyl group.

5. The compound of claim 4, wherein the phenyl group is substituted with a bromo group or a nitro group.

6. The compound of claim 1, wherein R¹ is a butynyl group.

7. The compound of claim 1, wherein R¹ has the formula VII 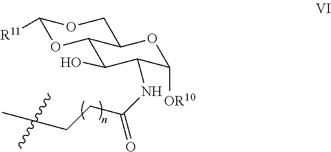
wherein R¹⁰ and R11 are, independently, an alkyl group or aryl group and n is from 1 to 5.

8. The compound of claim 7, wherein R¹⁰ is methyl, R¹¹ is phenyl, and n is 2.

9. The compound of claim 1, wherein R² is a cycloalkyl group or an alkyl ester of a carboxylic acid.

10. The compound of claim 1, wherein R² is a cyclohexyl group.

11. The compound of claim 1, wherein R² is an ethyl aceto group.

12. The compound of claim 1, wherein R³ and R⁴ are hydrogen.

13. The compound of claim 1, wherein R³ is hydrogen and R⁴ is a substituted or unsubstituted aryl group.

14. The compound of claim 1, wherein R⁵ is an aryl group, a substituted aryl group, an alkyl group, an amino sugar, or a protected amino sugar.

15. The compound of claim 1, wherein R⁵ is an α-methylbenzyl group or an unsubstituted or substituted benzyl group.

16. The compound of claim 15, wherein the benzyl group is substituted with a methoxy group.

17. The compound of claim 1, wherein R⁵ is glucosamine or a protected glucosamine.

18. The compound of claim 1, wherein the compound is
(a) N-benzyl-N-(2-(cyclohexylamino)-2-oxoethyl)pent-4-ynamide.
(b) N-benzyl-4-bromo-N-(2-(cyclohexylamino)-2-oxoethyl)benzamide;
(c) N-benzyl-N-(2-(cyclohexylamino)-2-oxoethyl)-4-nitrobenzamide;
(d) N-(4-methoxybenzyl)-4-bromo-N-(2-(cyclohexylamino)-2-oxoethyl)benzamide;
(e) ethyl 2-(2-(N-(4-methoxybenzyl)-4-bromobenzamido)acetamido)acetate.
(f) 4-bromo-N-(2-(cyclohexylamino)-2-oxoethyl)-N-(8-hydroxy-6-methoxy-2-phenyl-hexahydropyrano[3,2-d][1,3]dioxin-7-yl)benzamide; or
(g) ethyl-2-(2-(4-bromo-N-(8-hydroxy-6-methoxy-2-phenyl-hexahydropyrano[3,2-d][1,3]dioxin-7-yl)benzamido)acetamido)acetate.

19. The compound of claim 1, wherein the compound has the structure

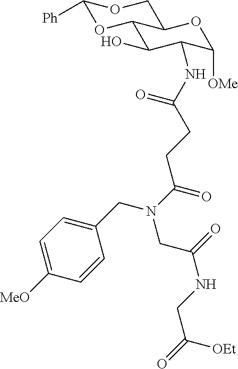 [SMILES: CCOC(=O)CNC(=O)CN(Cc1ccc(OC)cc1)C(=O)CCC(=O)NC1C(OC)OC2COC(c3ccccc3)OC2C1O]

20. A method for making a compound of formula (I) of claim 1 comprising reacting the following components:

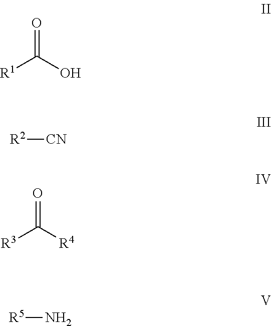

21. The method of claim 20, wherein the method comprises admixing the components in the following order:
(a) admixing compound V with compound IV to produce a first admixture;
(b) admixing compound II with the first admixture to produce a second admixture; and
(c) admixing compound III with the second admixture to produce the compound having the formula I.

22. A gel comprising a compound of claim 1.

23. The gel of claim 22, further comprising a bioactive agent or a catalyst immobilized on the gel.

24. A method for producing a gel comprising adding the compound of claim 1 to a solvent

25. The method of claim 24, wherein the solvent comprises water, an organic solvent, or a mixture thereof.

26. The method of claim 24, wherein the solvent comprises an alcohol.

27. The method of claim 24, wherein the solvent comprises a mixture of an alcohol and water.

28. The method of claim 24, wherein the solvent comprises a hydrocarbon.

29. The method of claim 24, wherein the solvent comprises a mixture of a hydrocarbon and water.

30. The method of claim 24, wherein the solvent is hexane, water, or ethanol.

31. The method of claim 24, wherein the solvent mixture is an ethanol:water mixture.

32. The method of claim 24, wherein the solvent mixture is a DMSO:water mixture.

33. A method for separating one or more chiral compounds, the method comprising contacting the one or more chiral compounds with a gel comprising a compound of claim 1, wherein the compound is optically active.

34. The method of claim 33, wherein the chiral compounds is a peptide, protein, DNA, or a glycan.
